(19) United States
(12) Patent Application Publication
(10) Pub. No.: US 2002/0187142 A1
(43) Pub. Date: Dec. 12, 2002

(54) Bispecific molecules cross-linking ITIM and ITAM for therapy

(76) Inventors: David Thomas, Houston, TX (US); Sunny Tam, Missouri City, TX (US)

Correspondence Address: TANOX, INC., 10301 STELLA LINK, HOUSTON, TX, 77025, US

(21) Appl. No.: 10/213,216

(22) Filed: Aug. 6, 2002

Related U.S. Application Data
(63) Continuation of application No. 09/440,310, filed on Nov. 17, 1999, now abandoned.
(60) Provisional application No. 60/108,816, filed on Nov. 17, 1998.

(51) Int. Cl. A61K 39/395; C12N 5/06; C12P 21/02; C07K 16/40

(52) U.S. Cl. 424/130.1; 435/326; 435/320.1; 435/69.1; 530/387.1

(57) ABSTRACT

The invention includes bispecific molecules capable of cross-linking ITAM and ITIM receptors on a cell in order to inhibit cell activation, as well as gene therapy approaches using nucleotides encoding such bispecific molecules for expression in vivo. One example of an ITAM/ITIM receptor pair is FcεFRI and HM18, and another is FcεFRI and FcγRII. Cross-linking of these receptors with a bispecific molecule of the invention would lead to inhibition of the release of allergic mediators and amelioration of the symptoms of allergic diseases.

### BACKGROUND OF THE INVENTION

IgE is well known as the mediator of immediate-type hypersensitivity allergic reactions, including allergic rhinitis (“hay fever”), extrinsic asthma, and food and drug allergies. In IgE-mediated allergic reactions, IgE, after it is secreted by B cells, binds through its Fc portion to the FcεRI receptors, which are present on the surface of basophils, mast cells and Langerhans cells. If the IgE bound to the surface of these cells now contacts and binds an allergen, this causes a cross-linking of the bound IgE molecules and hence the underlying receptors, and triggers the release of pharmacologic mediators, such as histamine, serotonin, leukotrienes and the slow-reacting substance of anaphylaxis. These mediators cause the pathologic manifestations of allergic reactions.

Cross-linking of ITAM (immunoreceptor tyrosine-based activation motif) with ITIM (immunoreceptor tyrosine-based inhibition motif) may cause negative regulation of cell activation. In fact, it has been demonstrated in animal models that cross-linking of IgE bound to FcERI (ITAM) with gp49 (ITIM) inhibits release of mast cell mediators of allergy. International Patent Application WO 98/09638 discusses a rat antibody which targets gp49 (designated mAb B23.1) on the surface of murine mast cells. It was demonstrated that coligation of the FcεRI and gp49 on the surface of mast cells suppresses FcεRI mediated exocytosis, as evidenced by the release of the secretory granule mediator ε-hexosaminidase and the generation of the membrane derived pro-inflammatory lipid mediator leukotriene (LT) C4.

The coligation in this International patent application was accomplished using an F(ab′)₂ antibody fragment targeting the light chains of both the B23.1 antibody and rat IgE. The mast cells were primed with mAb B23.1 and rat IgE, and then the F(ab′)₂ fragment was added. Inhibition of exocytosis as compared with controls was evident. This inhibitory effect is believed to be caused by inhibition of the signal transduction cascade that otherwise leads to the release of such mast cell mediators.

Inhibition of mast cell allergic mediators through cross-linking of FcεRI with gp49 (or the human equivalent, HM18) offers new potential treatments for IgE-mediated allergic diseases. Cross-linking of other ITIM and ITAM receptors, which could inhibit activation of a number of cell types carrying these receptors, could provide treatments for a number of diseases and conditions where cellular activation is a disease component.

### SUMMARY OF THE INVENTION

The invention includes bispecific molecules capable of cross-linking ITAM and ITIM receptors on a cell in order to inhibit cell activation, as well as gene therapy approaches using nucleotides encoding such bispecific molecules for expression in vivo. One example of an ITAM/ITIM receptor pair is FcεRI and HM18. Cross-linking of these receptors with a bispecific molecule of the invention would lead to inhibition of the release of allergic mediators and amelioration of the symptoms of allergic diseases.

The bispecific molecules include bispecific antibodies or diabodies, having one specificity derived from an anti-ITAM antibody (including FcεRI) and the other from an anti-ITIM antibody (including HM18). One could also indirectly cross-link the ITAM/ITIM receptors with bispecific molecules binding to molecules which bind to one or to both of these receptors. Examples include a bispecific molecule with one arm binding IgE (which in turn binds to FcεRI) or to an allergen (which is bound by the IgE bound to FcεRI), and the other arm binding to HM18.

A number of different bispecific molecules or antibodies can be used, one example being two single chain Fv antibodies linked together. Such bispecific antibodies (diabodies) are described in U.S. Pat. No. 5,534,254 (Creative Biomolecules, Inc.).

When administered in vivo, these bispecific molecules bind, directly or indirectly, to an ITAM (e.g.; FcεRI) and to an ITIM (e.g. HM18), which are both present on mast cell surfaces. This binding will likely result in inhibition of cell activation. In the case where the bispecific molecule binds FcεRI and HM18, this would inhibit exocytosis and release of the pharmacologic mediators of allergic diseases.

### BRIEF DESCRIPTION OF THE DRAWINGS

FIG. 1 shows the percentage of inhibition of histamine release from mast cells using the reagents and antibodies as indicated below the X axis.

FIG. 2 shows the inhibition of β-hexosaminidase release from C57BL/6 mouse mast cells upon crosslinking of FcεRI and FcγRII receptors by a rabbit IgG anti-HSA.

FIG. 3 shows the inhibition of β-hexosaminidase release from C57BL/6 mouse mast cells upon crosslinking of FcεRI and FcγRII receptors by a bispecific 2.4G2-anti-DNP antibody.

FIG. 4 shows the inhibition of passive cutaneous anaphylaxis in Balb/c mice upon crosslinking of FcεRI and FcγRII receptors by a bispecific 2.4G2-anti-DNP antibody.

### MAKING AND USING THE INVENTION

One embodiment of the bispecific molecules of the invention is formed by conjugating two single chain antibodies, one derived from an antibody specific for an ITAM (e.g., FcεRI) and the other from an antibody specific for an ITIM (e.g., HM18). Another embodiment is a fusion protein including a monoclonal antibody to FcεRI, or a fragment thereof, and an antibody to HM18, or a fragment thereof. The monoclonal antibodies used to form the bispecific molecules include, in whole or in part, as appropriate, chimeric antibodies, humanized antibodies, human antibodies, single-chain antibodies and fragments, including Fab, F(ab′)₂, Fv and other fragments which retain the antigen binding function of the parent antibody. Single chain antibodies (“ScFv”) and the method of their construction are described in U.S. Pat. No. 4,946,778.

Chimeric antibodies are produced by recombinant processes well known in the art, and have an animal variable region and a human constant region. Humanized antibodies correspond more closely to the sequence of human antibodies than do chimeric antibodies. In a humanized antibody, only the complementarity determining regions (CDRs), which are responsible for antigen binding and specificity, are non-human derived and have an amino acid sequence corresponding to the non-human antibody, and substantially all of the remaining portions of the molecule (except, in some cases, small portions of the framework regions within the variable region) are human derived and have an amino acid sequence corresponding to a human antibody. See L. Riechmann et al., Nature; 332: 323-327 1988; U.S. Pat. No. 5,225,539 (Medical Research Council); U.S. Pat. Nos. 5,585,089; 5,693,761; 5,693,762 (Protein Design Labs, Inc.).

Human antibodies can be made by several different methods, including by use of human immunoglobulin expression libraries (Stratagene Corp., La Jolla, Calif.; Cambridge Antibody Technology Ltd., London, England) to produce fragments of human antibodies (V_{H}, V_{L}, Fv, Fd, Fab, or (Fab′)₂) and use of these fragments to construct whole human antibodies by fusion of the appropriate portion thereto, using techniques similar to those for producing chimeric antibodies. Human antibodies can also be produced in transgenic mice with a human immunoglobulin genome. Such mice are available from Abgenix, Inc., Fremont, Calif., and Medarex, Inc., Annandale, N.J. In addition to connecting the heavy and light chain Fv regions to form a single chain peptide, Fab can be constructed and expressed by similar means (M. J. Evans et al., J. Immunol. Meth., 184: 123-138 1995).

All of the wholly and partially human antibodies described above are less immunogenic than wholly murine or non-human-derived antibodies, as are the fragments and single chain antibodies. All these molecules (or derivatives thereof) are therefore less likely to evoke an immune or allergic response. Consequently, they are better suited for in vivo administration in humans than wholly non-human antibodies, especially when repeated or long-term administration is necessary, as may be needed for treatment with the bispecific antibodies of the invention.

U.S. Pat. No. 5,534,254 (Creative Bimolecules, Inc.) describes several different embodiments of bispecific antibodies, including linking single chain Fv with peptide couplers, including Ser-Cys, (Gly)₄-Cys, (His)₆-(Gly)₄-Cys, chelating agents, and chemical or disulfide couplings including bismaleimidohexane and bismaleimidocaproyl. Another embodiment is a dimer having single chain FvL₁ and FvH₂ linked and FvH₁ linked with FvL₂ All such linkers and couplings can be used with the bispecific antibodies of the invention.

The bispecific molecules of the invention are administered as a pharmaceutical composition at a dosage effective to inhibit mast cell exocytosis. The effective dosage can be readily determined in routine human clinical trials or by extrapolation from animal models.

Typically, the pharmaceutical composition is administered by injection, either intravenously, subcutaneously or intraperitoneally. It may also be possible to obtain compositions which may be topically or orally administered, or which may be capable of transmission across mucous membranes.

Before administration to patients, formulants and excipients, well known in the art, are preferably added to the pharmaceutical composition. Additionally, pharmaceutical compositions can be chemically modified by covalent conjugation to a polymer to increase their circulating half-life. Polymers, and methods to attach them to peptides, are shown in U.S. Pat. Nos. 4,766,106; 4,179,337; 4,495,285; and 4,609,546, and include polyoxyethylated polyols and PEG.

Making monoclonal antibodies against mouse gp49 (the mouse counterpart of HM18) is described in Katz et al., 1989, J. Immunol. 142:919-926. Monoclonal antibodies against human HM18 would be made using analogous methods and techniques, with the human HM18 polypeptide described in International Patent Application WO 98/09638 as the immunogen. Monoclonal antibodies against FcεRI can be made using similar techniques, using a recombinant peptide representative of the sequence of the receptor. These peptides can be linked to a carrier, for example, keyhole limpet hemocyanin, to increase the immunogenicity and the production of antibodies to the immunogen.

Following production of antibody candidates, the antibodies would be screened against mast cells bearing HM18 and FcεRI or against recombinant versions of these receptors. Similar techniques could be used to generate bispecific antibodies capable of indirectly cross-linking HM18 and FcεRI, e.g., bispecific antibodies with one arm binding to IgE or an allergen, and the other arm binding to HM18.

Bispecific antibodies against other ITAM/ITIM receptor pairs (or which are capable of indirectly cross-linking such ITAM/ITIM receptor pairs) could be made using similar techniques, with an appropriate ITAM and ITIM used as immunogens for the mice. The antibody candidates would be screened against the receptor pair, or, if appropriate, the indirect linker(s).

Other bispecific molecules, including peptides, DNA, RNA, other organic molecules or homologues or analogues thereof, can also be made against any ITAM/ITIM receptor pair, or, an indirect linker(s) thereto. Activating receptors include BCR, FcγRI, FcγRIIA, FcαRI, FcγRIIIA and TCR. These ITAM receptors are carried on a variety of human cells and could therefore be regulated by interaction with the appropriate ITIM receptor. The bispecific molecule candidates can then be screened for binding to the designated ITAM/ITIM receptor pair, or the indirect linker(s), using conventional procedures.

One can also express any of the diabodies of the invention, or other bispecific proteins of the invention, with gene therapy techniques. Gene constructs that direct the expression in vivo of the diabodies or bispecific proteins can be administered by an appropriate vector system, including a retrovirus, an adenovirus, a parvovirus or any other vector permitting cellular transfer of the gene constructs, or by incorporation of the gene construct into liposomes with or without the viral vector. In the alternative, such nucleotides can be used to transfect cells ex vivo, using known methods including electroporation, calcium phosphate transfection, micro-injection, or incorporation of the gene constructs into liposomes followed by transfection. The cells are then introduced into the patient for expression in vivo. Use of gene therapy for antibody expression is well known in the art. See, e.g., International Patent Application No. WO 98/31808.

### EXAMPLE I

### Generation of Single Chain Antibody Fragments from Monoclonal Antibodies to HM18 and to FcεRI.

The techniques described in this Example I can be used to generate a single chain antibody fragment (ScFv) of either the anti-HM18 or anti-FcεRI monoclonal antibodies.

Both the V_{H} and V_{L} region of the antibodies are amplified by PCR, followed by a second assembly PCR to connect both regions. Four primers are designed. The first contains a HindIII and SfiI restriction site for cloning purposes followed by a degenerated sequence annealing to the 5′ V_{H} region. The second contains a degenerate sequence for the 3′ part of the V_{H} region followed by a sequence encoding a ((Gly)₄Ser)₃ linker and the 5′ part of the V_{L} regions. The third is a degenerated primer having homology with the 5′ part of the V_{L} region, while the last primer contains a NotI restriction site and anneals to the 3′ part of the V_{L} region.

As a template for this PCR reaction, one can use a plasmid containing the V_{H} or V_{L} regions of the antibody of interest. The cDNA obtained in this PCR step is gel purified and used in an assembly PCR resulting in the linkage of the V region through the ((Gly)₄Ser)₃ linker. Subsequently the single chain construct obtained is digested with the restriction enzymes HindIII and NotI, followed by ligation in pGEM-13Zf (Promega, Madison, USA). The ligation is transformed in DH5α and plated on LB plates. By sequencing of several clones, a correct ScFv clone is found.

### EXAMPLE II

### Construction of Bispecific Diabody Molecules Capable of Binding to HM18 and FcεRI

Bispecific bivalent molecules can be generated by shortening the flexible linker sequence in the anti-HM18 ScFv and in the anti-FcεRI ScFv, from fifteen residues to five (Gly₄Ser) and by cross-pairing the variable heavy and light chain domains from the two single chain Fv fragments with the different antigen recognition. The construction is preferably performed in three steps. The light chain variable fragments are exchanged in the ScFv constructs from an anti-HM18 ScFv and an anti-FcεRI ScFv by using restriction enzyme sites located in the 5′-end and just outside the 3′-part of the light chain variable gene. In the following step the 15-residue linker of the chimeric construct V_{H}-a HM18/15AA-linker/V_{L}-a- FcεRI is replaced by the 5 residue linker (Gly₄Ser) by using sites located in the 3′-part of V_{H} and the 5′-part of V_{L}. Finally, a chimeric cassette is combined in the vector pUC119-fabsol (a pUC119 derivative similar to pUC119His6mycXba (Low et al., J. Mol. Biol. 260:359 (1996)), but with all ApaII-sites in the vector backbone deleted by in vitro mutagenesis) containing a bi-cistronic expression cassette. A diabody producing clone containing both ScFv-cassettes is identified and used for expression of the recombinant diabody molecule.

### EXAMPLE III

### Other Bispecific Molecules

In addition to diabodies, it is possible to construct other bispecific molecules capable of coligating HM18 and FcεRI, and suitable for treatment of IgE-mediated allergic diseases in accordance with the teachings of the invention. Such bispecific molecules can be small molecules, antibody homologues or analogues, nucleotides or other molecules capable of coligating HM18 and FcεRI. Such bispecific molecules can be isolated from libraries or otherwise by screening against HM18 and FcεRI, and conducting any further in vitro functional assays, as necessary, including the cellular assays described below.

### EXAMPLE IV

### Cellular Assays to Screen for Non-Activating High Affinity Antibodies Against IgE or FcεRI Receptor and HM18 Antibodies

Human FcεRI receptor (Invitrogen Inc. or Beth Israel Hospital) and human HM18 receptor (Brigham & Women Hospital) can be co-transfected into the rat mast cell line, RBL-2H3 (ATCC collection). Briefly, transfected cells are resuspended in RPMI 1640 medium supplemented with 10% FCS at 1×10⁶ cells/ml and incubated at 37° C. for 1 hour with 2 uCi/ml [³H] serotonin (Amersham Corp.). The cells are washed and reincubated for another hour at 37° C. and transferred to 96-well microculture plates at 2×10⁵ cells/well. Cells are then treated with individual bispecific hybridoma supernatant or carrier coupled diabody specific for human FcεRI and HM18 receptors and left for 1 hour. Before challenge with human IgE and IgE crosslinking reagent for 30 minutes, the attached cells are washed and warmed for 15 minutes at 37° C. Reactions are terminated by adding 50 ul of ice-cold medium and by placing plates on ice. 50 ul of supernatants are mixed with 1 ml of emulsifier safe scintillation liquid and counted in a Beckman counter. The percentage of serotonin release is calculated using as 100% the cpm obtained from the same number of pulsed cells lysed in 0.5% SDS and NP-40. The diabodies that can inhibit maximum serotonin release would be the choice for further characterization.

For further testing of the selected diabody constructs, a secondary confirmatory human cord blood derived mast cell culture could be used. Briefly, cultured human mast cells can be raised from commercially available CD34+ purified human umbilical cord blood mononuclear cells through the addition of 80 ng/ml of rhSCF, 50 ng/ml rhIL-6 and 5 ng/ml rhIL-10 for 6 to 8 weeks. The confirmed mast cell population after FACs sorting will be plated into 96-well microculture plates. Cells are then treated with selected diabody supernatants for 1 hour. Before challenge with human IgE and IgE crosslinking reagent for 30 minutes, the cells are washed and warmed for 15 minutes at 37° C. The histamine release into the media is measured with the ELISA detection system from Immunotech, Inc. The diabodies that can inhibit maximum histamine release would be the choice for further characterization.

### EXAMPLE V

### Reduction of Mast Cell Degranulation by Cross-Linkinq of the ITIM and ITAM Receptors

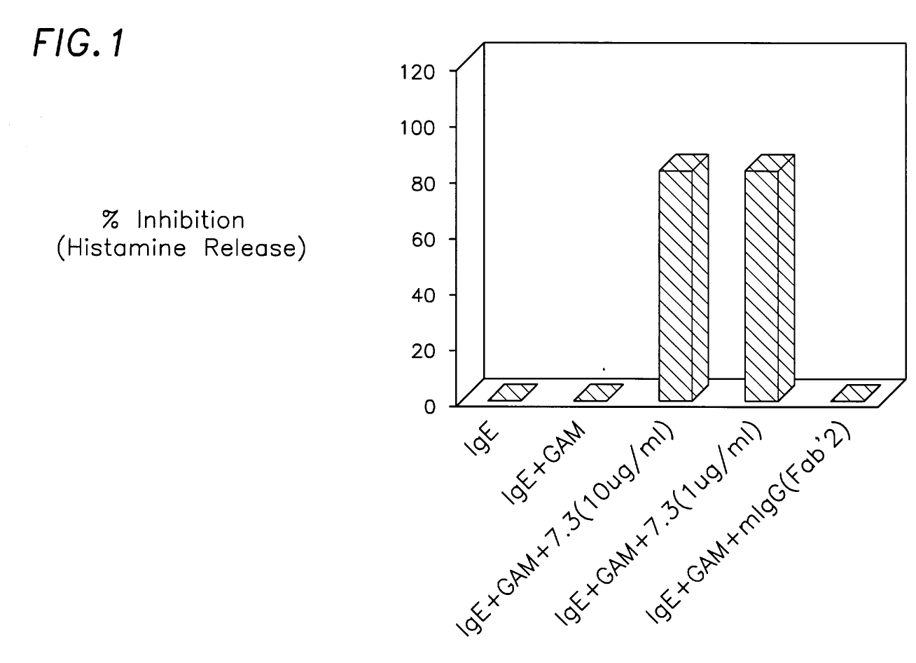

A monoclonal anti-human FcγRII antibody, clone 7.3, significantly diminished IgE induced human mast cell degranulation by more than 90% when the IgE receptor is crosslinked to the inhibitory FcγRII receptor by the goat anti-mouse antibody (GAM). This is illustrated in the amount of histamine released from the IgE+GAM group versus the IgE+GAM+7.3 group. An isotype matched mouse IgG Fab′2 was used as a control, showing the importance of crosslinking the inhibitory receptor FcγRII to the IgE receptor (see FIG. 1). The experiment is described briefly below.

CD34+ cells were isolated from human cord blood using Dynal CD34 coated beads. The isolated cells were cultured in the presence of 80 ng/ml of recombinant human stem cell factor, 50 ng/ml of recombinant human interleukin 6 and 5 ng/ml of recombinant human interleukin 10 for about 8 weeks. For each group, 1×10⁵ human mast cells were treated with mouse IgE (10 μg/ml), mouse IgE+mouse anti-human FcγRII antibody, clone 7.3 (1 or 10 μg/ml), or isotype matched mouse IgG Fab′2 (10 μg/ml) for the first hour of incubation at 37° C. Subsequently, the cells were washed twice with PBS before applying the goat anti-mouse Fab′2 (GAM, 10 μg/ml) to the selected groups for another hour of incubation at 37° C. The histamine release into the supernatant from each treatment group was tested with the human histamine ELISA kit from Immunotech, Inc.

FIG. 1 shows histamine release inhibition of human cord blood mast cells upon crosslinking of FcεRI and FcγRII receptors. The percentage of inhibition was the comparison of the amount of histamine release (in nM) between the crosslinked groups versus the positive control group (IgE+GAM) in lane 2 in FIG. 1.

### EXAMPLE VI

### Mast Cell Inhibition with a Bispecific Rabbit IgG

Additional cellular experiments of the ITAM/ITIM concept were done with two forms of antibodies: 1) a bispecific single antibody; 2) a presumably bispecific rabbit IgG.

Crosslinking of FcεRI (ITAM) and FcγRII (ITIM) on a mouse mast cell line, C-57, using a presumably bispecific rabbit IgG, confirmed the significance of ITAM/ITIM receptor co-ligation in repressing mouse mast cell activation and degranulation in a dose dependent manner (ranging from 20% to 74% inhibition). The variable region of the rabbit IgG could recognize the FcεRI via the antigen (DNP-HSA), and the Fc region predominantly only recognizes the FcγRII and RIII family receptors.

The protocol was that mouse mast cells (2×10⁵ cells/group) were incubated with IgE anti-DNP (1 μg/ml) for 45 minutes at 37° C. DNP-HSA (5 ng/ml) was incubated with rabbit IgG anti-HSA (at various concentrations, ranging from 0.01 to 100 μg/ml) for 15 minutes at 37° C. After washing the treated cells with Tyrode's buffer, cells were incubated with the IgG complexed antigen for another 45 minutes at 37° C. The β-hexosaminidase activity released into the supernatant was measured the hydrolysis of pNpp.

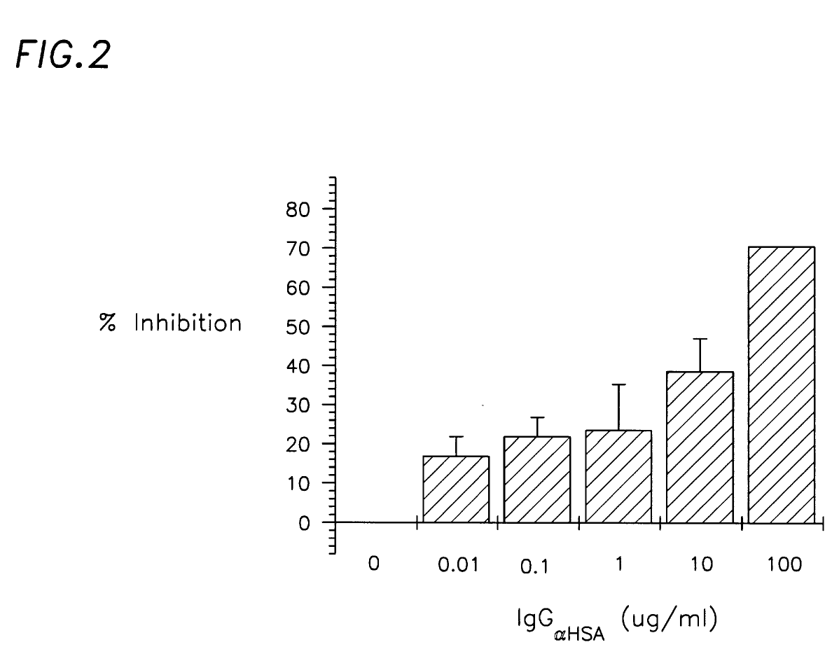

FIG. 2 shows the Inhibition of β-hexosaminidase release from C57BL/6 mouse mast cells upon crosslinking of FcεRI and FcγRII receptors by a rabbit IgG anti-HSA. The percent of inhibition was the comparison of the amount of β-hexosaminidase release (in nM) between the crosslinked groups (lanes 2 through 6) versus the positive control group (IgE+DNP-HSA, lane 1).

### EXAMPLE VII

### Inhibition with Another Bispecific Antibody

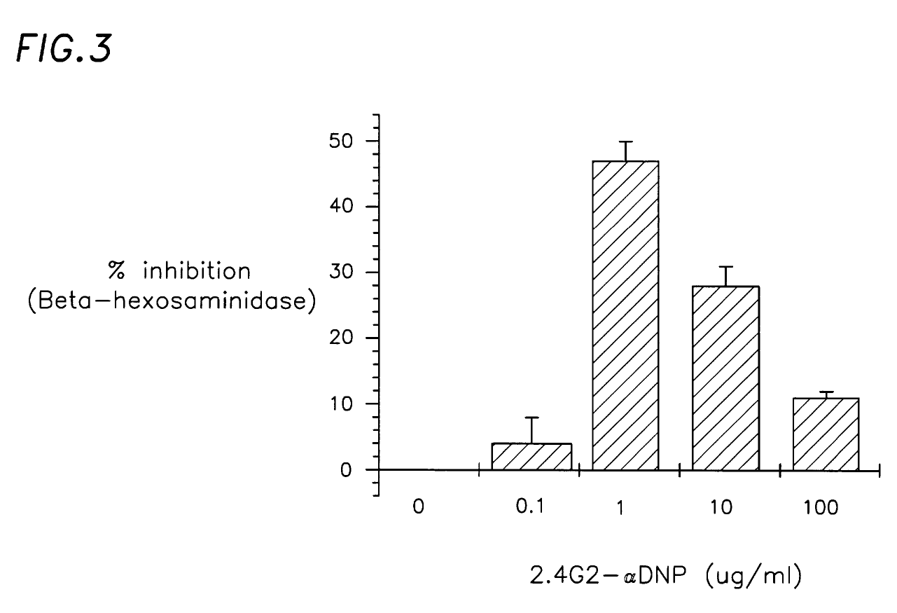

A chemically conjugated bispecific multivalent antibody that recognizes the di-nitrophenyl moiety (DNP) and mouse FcγRII/III receptors (clone 2.4G2) was obtained. Using this molecule at the optimal concentration of 1 μg/ml, a 50% inhibition of β-hexosaminidase release was demonstrated. At higher concentrations of 2.4G2-anti-DNP, such as 10 to 100 μg/ml, the inhibition is diminished. This is likely due to the increases in crosslinking the activatory receptors in addition to the inhibitory receptor, FcεRII B. The protocol for this experiment was that mouse mast cells (2×10⁵ cells/group) were incubated with IgE anti-DNP (1 μg/ml) both with and without 2.4G2-anti-DNP (at various concentrations, ranging from 0.1 to 100 μg/ml) for 45 minutes at 37° C. After washing with Tyrode's buffer, cells were incubated with the antigen, DNP-HSA (5 ng/ml) for another 45 minutes at 37° C. The β-hexosaminidase activity was measured by the hydrolysis of pNpp. FIG. 3 shows the inhibition of β-hexosaminidase release from C57BL/6 mouse mast cells upon crosslinking of FcεRI and FcγRII receptors by a bispecific 2.4G2-anti-DNP antibody. The percent of inhibition was the comparison of the amount of β-hexosaminidase release (in nM) between the crosslinked groups (lanes 2 through 5) versus the positive control group (IgE anti-DNP+DNP-HSA, lane 1).

### EXAMPLE IX

### Inhibition with a Passive Cutaneous Anaphylaxis Model

The ITAM/ITIM concept has been well documented with cellular models in the literature and demonstrated with human cultured mast cells, as described in Examples I to III. To demonstrate the impact and feasibility of aggregation of the ITAM/ITIM receptors in animals, the Segal's bispecific molecule was applied in a passive cutaneous anaphylaxis (PCA) animal model.

Using this model, a 30% to 40% inhibition of the anaphylactic response was demonstrated. Given the 2.4G2 antibody also recognizes many activatory RII and RIII receptors, this experimental data shows the dominance of the inhibitory receptors in vivo and the feasibility of the ITAM/ITIM crosslinking as a therapeutic. The protocol was that anesthetized Balb/c female mice (4 to 5 weeks old) were injected into the surface of the left ear 25 ng of anti-DNP IgE, and 25 ng of anti-DNP IgE plus the appropriate dose of the bispecific antibody (ranging from 0.25 to 1 μg/mouse) into the right ear. After 24 hours, 100 μg of DNP-HSA in 2% Evan's blue/PBS was delivered via the tail vein. Control animals (group 1) received only IgE in the left ear and the bispecific antibody alone in the right ear. After injecting the antigen for 45 minutes, the mice were sacrificed by cervical dislocation/CO₂. For quantification of the Evan's blue dye extravasation, the ears were removed, minced and incubated at 80° C. for 3 hours in 2 ml of formamide. The absorbance values were read at 610 nm and the standard deviation was calculated for each group of at least 3 animals.

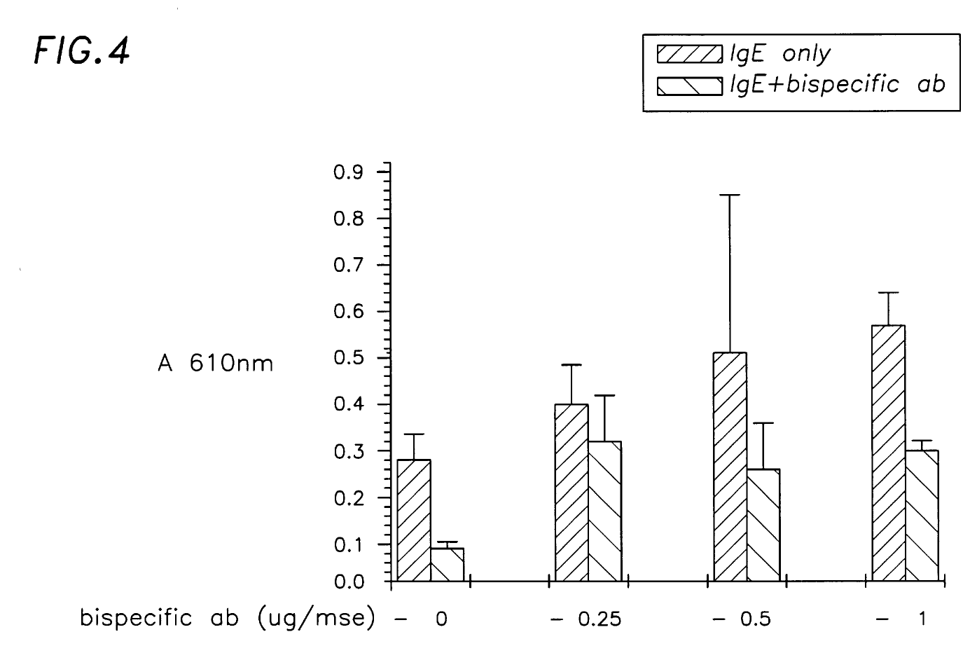

FIG. 4 shows the results, where inhibition of passive cutaneous anaphylaxis in Balb/c mice upon crosslinking of FcεRI and FcγRII receptors by a bispecific 2.4G2-anti-DNP antibody.

The examples and terms and expressions used above are exemplary only and not limiting, and the invention is defined only in the claims which follow, and includes all equivalents of the subject matter of the claims.

We claim:

1. A bispecfic molecule capable of directly or indirectly cross-linking an ITAM and an ITIM.

2. The bispecific molecule of claim 1 wherein one specificity is for the ITAM and the other is for the ITIM.

3. The bispecific molecule of claim 2 wherein one specificity is for HM18 and the other is for FcεRI.

4. The bispecific molecule of claim 2 wherein one specificity is for HM18 and the other is for IgE or an allergen.

5. The bispecific molecule of claim 2 wherein one specificity is for FcεRI and the other is for FcγRII.

6. The bispecific molecule of any of claims 1-5 which is a bispecific antibody or antigen binding fragment thereof, or a bispecific protein.

7. The bispecific molecule of claim 6 comprising antigen binding regions from two different antibodies or binding proteins.

8. Recombinant vectors comprising the nucleic acid sequences encoding the recombinant antibody fragments according to claim 6, and the necessary control elements to enable the expression of said recombinant antibody fragmentor in a host cell.

9. Recombinant vectors comprising the nucleic acid sequences encoding the recombinant antibody fragments according to claim 7, and the necessary control elements to enable the expression of said recombinant antibody fragments in a host cell.

10. A method for producing a bispecific molecule, comprising the steps of culturing a host cell which is transformed with a vector according to claim 8 under conditions enabling the expression of said bispecific molecule in said host.

11. A method for producing a bispecific molecule, comprising the steps of culturing a host cell which is transformed with a vector according to claim 9 under conditions enabling the expression of said bispecific molecule in said host.

12. A method of treating allergic diseases, comprising administering to a patient a therapeutically effective amount of a pharmaceutical composition including the bispecific molecules of any of claims 3 to 5.

13. A method of treating diseases or conditions associated with cell activation comprising administering gene constructs encoding bispecific antibodies or fragments thereof, or bispecific peptides, capable of directly or indirectly cross-linking an ITAM and an ITIM.

14. The method of claim 13 wherein one specificity is for the ITAM and the other is for the ITIM.

15. The method of claim 13 wherein the gene constructs are incorporated in a plasmid or a viral vector.

16. A method of treating allergic diseases comprising administering gene constructs encoding bispecific antibodies or fragments thereof, or bispecific peptides, capable of directly or indirectly crosslinking an ITAM and an HM18 and the other is for FcεRI.

17. The bispecific molecule of claim 16 wherein one specificity is for HM18 and the other is for IgE or an allergen.

18. Cells transfected or infected with the gene construct of any of claims 13 to 17.

19. The method of any of claims 13 to 17 wherein transfection or infection of the gene constructs is done ex vivo or in vivo.

20. The method of claim 19 wherein the transfection is done ex vivo by electroporation, calcium phosphate transfection, micro-injection or by incorporating the gene constructs into suitable liposomes.

21. The method of claim 19 wherein the infection is done in vivo or ex vivo by incorporating the gene constructs into a retrovirus, adenovirus or a parvovirus vector, or by incorporating the gene constructs, or the gene constructs with a viral or plasmid vector, into a suitable liposome.
